(19) United States
(12) Patent Application Publication
(10) Pub. No.: US 2009/0024034 A1
(43) Pub. Date: Jan. 22, 2009

(54) RELATIVE POSITION DETERMINATION MEDICAL ULTRASOUND SCANS

(76) Inventors: ROMAIN MOREAU-GOBARD, Palo Alto, CA (US); Gianluca Paladini, Skillman, NJ (US)

Correspondence Address: Siemens Corporation;Attn: Elsa Keller, Legal Administrator, Intellectual Property Department, 170 Wood Avenue South, Iselin, NJ, 08830, US

(21) Appl. No.: 12/182,983

(22) Filed: Jul. 30, 2008

Related U.S. Application Data
(63) Continuation-in-part of application No. 11/874,351, filed on Oct. 18, 2007.
(60) Provisional application No. 60/852,821, filed on Oct. 19, 2006.
(60) Provisional application No. 61/048,326, filed on Apr. 28, 2008.

(51) Int. Cl. A61B 8/14

(52) U.S. Cl. 600/443

(57) ABSTRACT

Using a conformal array or an ultrasound probe sequentially positioned at different locations, scans of overlapping regions are performed. A fiber optic sensor or light intensity based sensor determines the relative position of the probe or arrays for each of the scans. This relative position simplifies data correlation by limiting the search and/or is used to determine the relative position of the scans or data. The data may be aligned and combined.

### RELATED APPLICATIONS

The present patent document is a continuation-in-part of U.S. Pat. No. ______ (application Ser. No. 11/874,351), filed Oct. 18, 2007, which claims priority to provisional U.S. application 60/852,821, filed Oct. 19, 2006; and the present patent document claims priority to provisional U.S. application No. 61/048,326, filed Apr. 28, 2008, which are hereby incorporated by reference.

### FEDERALLY SPONSORED RESEARCH OR DEVELOPMENT

The U.S. Government has a paid-up license in this invention and the right in limited circumstances to require the patent owner to license others on reasonable terms as provided by the terms of grant no. DARPA05-01DBAC awarded by DARPA.

### BACKGROUND

The present embodiments relate to conformal arrays or scans at different positions. Multiple ultrasound transducers are embedded in a flexible surface or blanket. The blanket may be wrapped around or conform to the region of the body to be imaged or treated.

Ultrasound imaging is user-dependent and may require a sonographer with extensive experience and expertise in the field in order to produce repeatable results in image quality and interpretation of those images. Use of a blanket ultrasound device may reduce the required level of interaction with the sonographer. However, ultrasound may be used by non-specialists, for example in geographically remote areas (e.g., astronauts) or areas where an expert may not be available (e.g., a battlefield).

19% of sonographers have carpal tunnel syndrome because of the strong pressure they apply to the patient with the ultrasound probe. A conformal array may reduce direct contact with the patient. In ultrasound scanning workflow, the sonographer manually moves the probe back and forth to localize the region of interest. This localization may be done automatically in a blanket-ultrasound configuration, reducing the need for expertise.

For scanning with a single array, as the probe moves, the data is combined without prior knowledge. The success of such an operation greatly relies on the ability of the sonographer in acquiring high quality data. Large ultrasound blankets may scan the entire area of interest, but still combine data from different arrays. However, the combination may be computationally expensive.

### BRIEF SUMMARY

By way of introduction, the preferred embodiments described below include methods, systems, transducers, conformal arrays, computer readable media, and instructions for determining the relative position of ultrasound scans. Using a conformal array or an ultrasound probe sequentially positioned at different locations, scans of overlapping regions are performed. A fiber optic sensor or light intensity based sensor determines the relative position of the probe or arrays for each of the scans. This relative position simplifies data correlation by limiting the search and/or is used to determine the relative position of the scans or data. The data may be aligned and combined.

In a first aspect, a method is provided for determining relative position of ultrasound scans. First and second regions are scanned with first and second ultrasound transducers, respectively. A relative position of the first ultrasound transducer with the second ultrasound transducer is measured as a function of light intensity. Data from the scanning of the first region is spatially aligned with data from the scanning of the second region. The spatially aligning is a function of the relative position.

In a second aspect, a system is provided for determining relative position of ultrasound scans. A blanket flexibly connects a plurality of arrays. A fiber optic sensor connects with, or is adjacent to, each of the arrays of the plurality. A processor is operable to determine a bend of the blanket between the arrays as a function of output of the fiber optic sensor.

In a third aspect, a medical ultrasound transducer is provided. Fiber optic strands connect with a conformal array of transducer elements. A sensor is operable to determine bend of the conformal array as a function of light transmitted in the fiber optic strands.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for high intensity ultrasound path determination;

FIG. 2 is a perspective view of a blanket transducer arrangement for ultrasound imaging and high intensity focused ultrasound therapy according to one embodiment;

FIG. 3 is a flow chart diagram of one embodiment of a method for high intensity ultrasound path determination;

FIG. 4 is a graphical representation of one example of path determination;

FIG. 5 is an example medical image showing two selected paths;

FIGS. 6A-F show example effects for high intensity focused ultrasound beam characteristics; and

FIG. 7 is a perspective view of a blanket transducer arrangement with a fiber optic sensor.

### DETAILED DESCRIPTION OF THE DRAWINGS AND PRESENTLY PREFERRED EMBODIMENTS

A fiber optic sensor determines the position of different scans. For example, fiber optic strands in a conformal array are used to determine the relative positions of different arrays. When the conformal array conforms to the body being examined, the arrays alter position. Light intensity in the fiber optic strands indicates the position and/or orientation of the different arrays. The position information is used to spatially align data from the different arrays.

Data correlation may be used, but the search space may be narrowed by the position information from the fiber optic sensor. Where the fiber optic sensor has poor resolution as compared to the ultrasound sampling for a scan, the position from the fiber optic sensor may be used to initialize or limit the data correlation positioning. Using the fiber optic sensor may reduce the computations needed for data correlation. Where the conformal device includes arrays not used for a given patient (e.g., a small arm may be surrounded by six of the available 10 arrays), the fiber optic sensor may determine by position of the unused arrays that they are unused. Scans using those arrays may be avoided, reducing complexity for aligning the data. The fiber optic sensor may not need calibration for operation since relative position is measured in some embodiments.

In one example embodiment, multiple transducers are wrapped around or lay along a portion of a patient. By providing the transducers in one device, such as a semi-flexible blanket, movement of the device may be limited or the therapy and imaging transducers may be subject to the same movement, easing alignment.

At least some of the transducers in this example may be used for volume imaging and therapy with ultrasound. As volumes are acquired from each transducer, post-processing software aligns and combines the data representing the scanned volumes to make one complete volume. The alignment is performed using, at least in part or solely, output from a fiber optic position sensor. The compounded volume may have improved image quality because of spatial compounding. The software and/or user input detect a bleeder or other treatment location. Once the area of interest has been identified in the volume, the software identifies which transducers to use for therapy. Obstructions, tissue characteristics, or other factors are used to select from the possible paths. For example, the volume data is examined to identify desired or undesired acoustic paths. As another example, test transmissions are performed along possible paths, and the returning echo signals indicate the desirability of the tested paths. After selection of one or more paths, a homeostasis beam is transmitted.

The acoustic data representing the selected paths may be used to compute the dose to seal the bleeding vessel. The localization information and the power dose are determined from acoustic data. The entire detection of internal bleeding and treatment may be automatic.

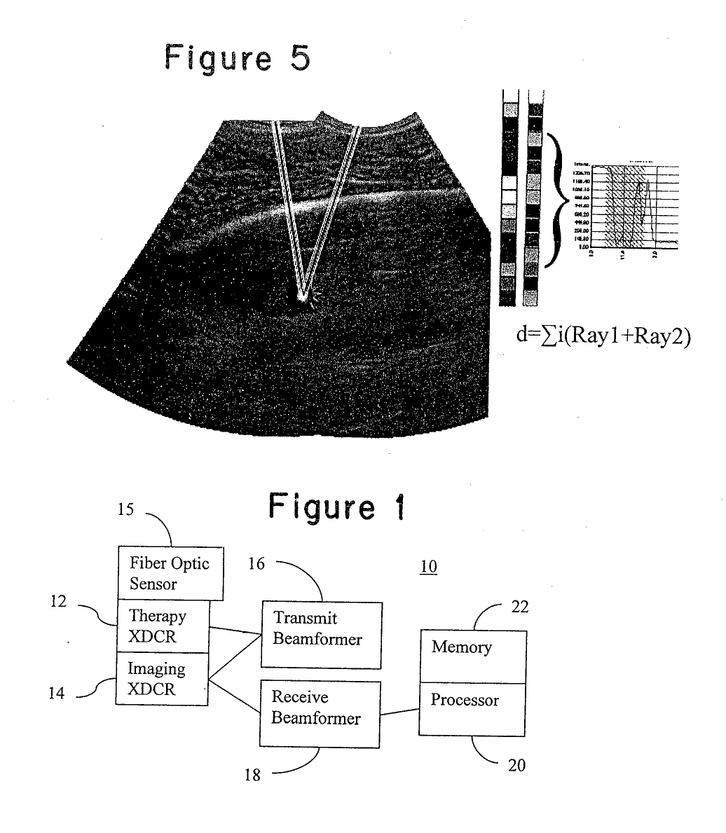

FIG. 1 shows a system 10 for determining relative position of ultrasound scans. The system 10 includes a therapy transducer 12, an imaging transducer 14, a fiber optic sensor 15, a transmit beamformer 16, a receive beamformer 18, a processor 20, and a memory 22. Additional, different, or fewer components may be used. For example, the therapy and imaging transducers 12, 14 may be a same device. As another example, more transducers of either type may be provided. Only imaging or only therapy transducers 12, 14 may be provided. In another embodiment, only a single transducer array is provided. In another example, a display is provided. Different transmit beamformers 16 may be used for the different types of transducers 12, 14.

In one embodiment, the system 10 is part of an ultrasound imaging and/or therapy system. The system 10 may be for operation with one or more of the transducers 12, 14 internal or external to the patient. A cart imaging system, computer, workstation, or other system may be used. In another embodiment, the system 10 is portable, such as for carrying by medics, soldiers, emergency response personnel, or others. The portable system 10 weighs from 1-30 kg.

The transducers 12, 14 with the fiber optic sensor 15 are a medical ultrasound transducer. The fiber optic sensor 15 detects the relative or absolute position of one or both transducers 12, 14. The transducers 12, 14 each include one or more elements. For example, each transducer 12, 14 include an array of elements. The medical ultrasound transducer includes the plurality of arrays.

The therapy transducer 12 is any now known or later developed transducer for generating high intensity focused ultrasound from electrical energy. A single element may be provided, such as where focus is provided mechanically by movement or a lens. A plurality of elements in a one or multi-dimensional array may be used, such as an array of N×M elements where both N and M are greater than one for electric based focusing or steering.

The element or elements are piezoelectric, microelectromechanical, or other transducer for converting electrical energy to acoustic energy. For example, the therapy transducer 12 is a capacitive membrane ultrasound transducer.

The therapy transducer 12 is operable from outside a patient. For example, the therapy transducer 12 is a probe or other device held against the patient's skin. The therapy transducer 12 is handheld, positioned by a device, or strapped to the patient. In other embodiments, the therapy transducer 12 is in a probe, catheter or other device for operation from within a patient.

In one embodiment, only one therapy transducer 12 is provided. In other embodiments, a plurality of therapy transducers 12 is provided. For example, a plurality of two-dimensional arrays of elements is used for transmitting from different locations to a treatment region. Alternatively, no therapy transducer 12 is provided.

The imaging transducer 14 is the same or different type, material, size, shape, and structure than the therapy transducer 12. For example, each of the one or more imaging transducers 14 includes a multi-dimensional array of capacitive membrane ultrasound transducer elements. The imaging transducer 14 is any now known or later developed transducer for diagnostic ultrasound imaging. The imaging transducer 14 is operable to transmit and receive acoustic energy. In alternative embodiments, no imaging transducer 14 is provided.

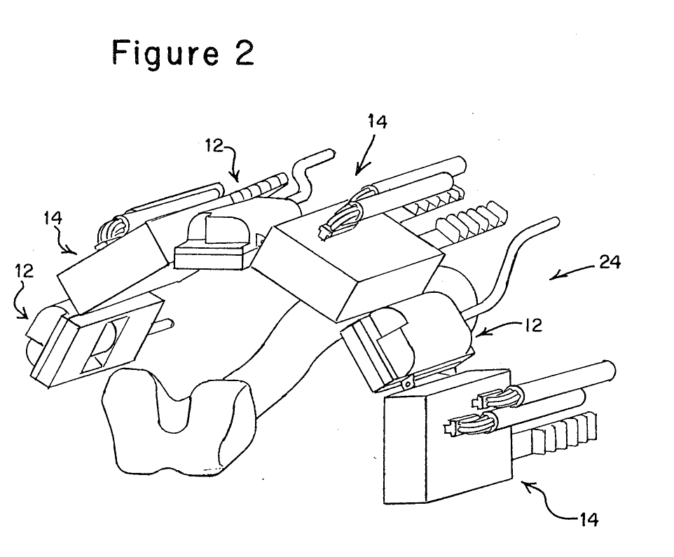

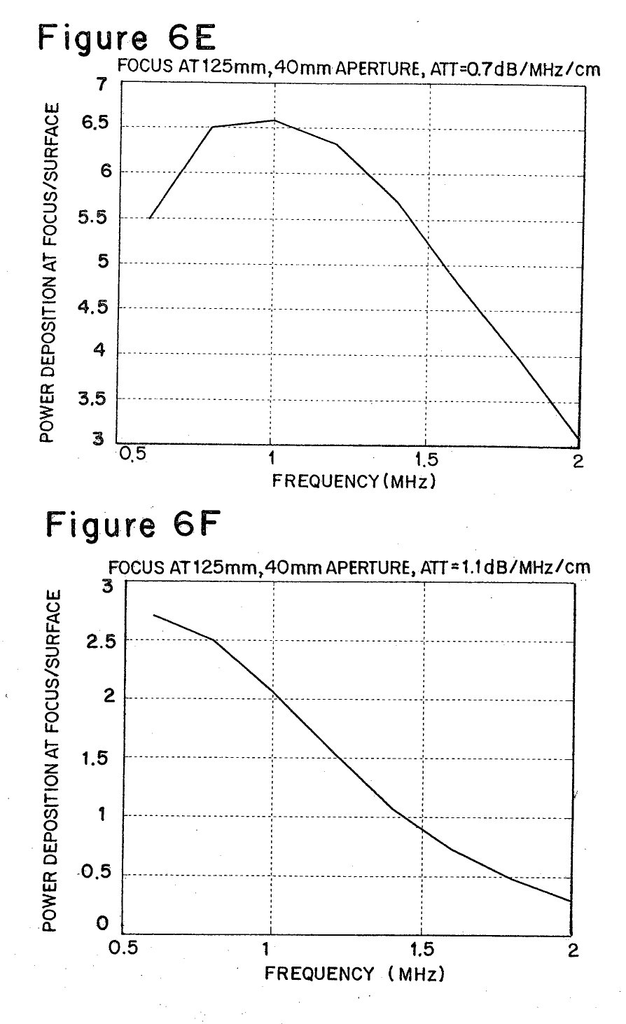

In one embodiment, the therapy and imaging transducers 12, 14 are in a blanket 24. According to specific embodiments, the blanket 24 is plastic, metal, fabric, or other material for rigidly, semi-rigidly and/or flexibly holding the plurality of transducers 12, 14 with or without the beamformers 16, 18, and/or processor 20. For example, FIG. 2 shows a blanket 24 with a plurality of transducers 12, 14. Hinges, other structure, or an outer casing interconnect the transducers 12, 14. For example, hinges connect the transducers 12, 14. One or more sets of transducers may be more rigidly connected. FIG. 7 shows another example of the blanket 24. The blanket 24 is shaped as a cuff, such as including a balloon or expandable chamber. Alternatively, Velcro® fasteners, buttons, buckles or other connector allow adjustable placement of the cuff around limbs or body portions of different sizes. In other embodiments, the blanket 24 conforms to a portion of the body without enclosing or surrounding the portion, such as being a patch that may conform to the torso of a patient.

The blanket 24 and transducers 12, 14 are a conformal array. The conformal array flexibly interconnects the transducers 12, 14 and/or elements of a same array. In the example of FIG. 7, the blanket 24 includes several rigid acoustic array panels 25. The array panels 25 are interlinked with a bendable and slightly expandable material, such as a spandex material. As shown, the array panels 25 may be connected such that a different number of panels 25 are to be used. The maximal circumference of a man's thigh may be about 80 cm. For the same device to be used on a woman's biceps, a lesser circumference is needed. The selection of the number of panels 25 and/or the flexibility of the blanket 24 allows use as a cuff on different patients and locations. The blanket 24 may be unrolled to be used against a torso.

Any amount of flexibility may be provided. More flexibility may allow for more overlap of the scan regions from the different arrays, providing more accurate alignment of data.

In the conformal array embodiment of FIG. 2, the blanket 24 includes every other transducer as an imaging transducer 14 and a therapy transducer 14. Other ratios and/or arrangements may be provided. One, more, or all of the transducers may be dual use devices, such as each transducer 12, 14 being for imaging and therapy. In one embodiment, each of the imaging transducers 14 is operable to electronically, or electronically and mechanically, scan in three dimensions for acquiring data representing a volume. The transducers 14 may be arranged such that, at least for deeper depths, the scan volumes of adjacent imaging transducers 14 overlap.

A covering, such as a fabric, plastic or other material, may relatively connect the transducers 12, 14. The blanket 24 is a cuff or other structure for wrapping around or resting on a patient. FIG. 2 shows the blanket 24 of transducers 12, 14 wrapped at least partially around a leg or arm. The ultrasound devices are embedded in a flexible surface, wrapped around the region of the body needing medical attention. This geometry may allow acquiring 360-degree images around a limb, or larger volumes than with a single array.

In another embodiment of a medical ultrasound transducer, a single array conforms by having elements flexibly connected together. The fiber optic sensor 15 detects the relative position of elements.

The spatial relationship between the transducers 12, 14, elements of an array, and/or different arrays is measurable. For example, pairs of the imaging and therapy transducers 12, 14 are fixedly connected together. A sensor measures the relative position between fixedly connected pairs. As another example, a sensor measures the relative motion between the imaging and therapy transducers 12, 14.

Any sensor may be used, such as magnetic position sensors, strain gauges, fiber optics, or other sensor. Alternatively or additionally, acoustic response from the arrays indicates the relative positions. Correlation of imaging data may indicate the spatial relationship between imaging transducers 14. The fiber optic sensor 15 is used in combination with other sensors and/or data correlation. Alternatively, the fiber optic sensor 15 is used without other position determination.

The fiber optic sensor 15 includes fiber optic strands and a sensor operable to determine the bend and/or twist of the fiber optic strands from light transmitted in the fiber optic strands. The fiber optic strands are light guides with a layer of transmissive material. The sensor detects the location through modulation of the intensity of light from one strand to another. In one embodiment, one of the fiber optic sensors 15 disclosed in U.S. Pat. No. 5,633,494 is used. Other now known or later developed fiber optic sensors may be used, such as the Shapetape sensor available from Measurand, Inc. Other fiber optic sensors include sensors used for real-time motion capture and/or data glove type devices for user input.

The fiber optic sensor 15 connects with or adjacent to the elements or arrays in the blanket 24. Glue, clips, positioning in a pocket, brackets, and/or other connections may be provided. In one embodiment, the fiber optic sensor 15 connects adjacent to the arrays with brackets. One or more fiber optic sensors 15 may be used. FIG. 7 shows two fiber optic sensors 15, one on each edge of the conformal array. The fiber optic sensors 15 are adjacent to each of the arrays, but may be connected adjacent fewer than all of the arrays, such as where one array is rigidly connected with another array. The fiber optic strands may be covered with a protective layer, such as being positioned within the blanket 24 and covered by a covering of the blanket 24. Alternatively, the fiber optic strands have their own protective covering and are on the outside of the blanket 24.

Where the conformal array is expandable, such as associated with stretchable material, the fiber optic sensor 15 may be connected to or adjacent the arrays with slack. A bend is built into the fiber optic sensor 15 when the blanket 24 is at a relaxed state. When the blanket 24 expands, the bend in the fiber optic strands may be lessened.

The fiber optic sensor 15 detects bending and/or twisting along the length of the fiber optic sensor 15. The position of the arrays or elements relative to the length of the fiber optic sensor 15 is known by the fixed attachment of the fiber optic sensor 15 to or adjacent to the arrays. The orientation and/or position of different locations along the fiber optic position sensor relative to other locations are determined by measuring light in fiber optic strands. The bending, and/or twisting of the fiber optic position sensor is measured, such as measuring at a time after the transducer is positioned adjacent an acoustic window. The relative position of the transducer at different acoustic windows may be determined.

The transmit beamformer 16 has a plurality of waveform generators, amplifiers, delays, phase rotators, and/or other components. For example, the transmit beamformer 16 has waveform generators for generating square or sinusoidal waves in each of a plurality of channels. The waveform generators or downstream amplifiers set the amplitude of the electrical waveforms. For imaging, the amplitude is set to provide scanning with acoustic beams below any limits on imaging amplitude. The amplitude may be set for high intensity focused ultrasound, such as higher than associated with imaging.

Relative delays and/or phasing of the waveforms focus the transmitted acoustic energy. By applying relatively delayed and/or apodized waveforms to different elements of a transducer, a beam of acoustic energy may be formed with one or more foci along a scan line. Multiple beams may be formed at a same time. For electronic steering, the relative delays establish the scan line position and angle relative to the transducer 12, 14. The origin of the scan line on the transducer 12, 14 is fixed or may be adjusted by electronic steering. For example, the origin may be positioned on different locations on a multi-dimensional array. The different origins result in different positions of the respective scan lines.

The receive beamformer 18 receives electrical signals from the imaging transducer 14. The electrical signals are from different elements. Using delay and sum beamforming, fast Fourier transform processing, or another process, data representing different spatial locations in a plane or in a volume is formed. One, a few, or many transmission and reception event(s) may be used to scan a volume with the imaging transducer 14. For example, plane wave transmission and reception is used for scanning a volume. Multiple beam reception with or without synthetic beam interpolation may increase the speed of volume scanning with delay and sum beam formation. In alternative embodiments, a two-dimensional plane or scan lines are scanned instead of a three-dimensional volume.

The beamformed data is detected. For example, B-mode detection is provided. In another example, Doppler power, velocity, and/or variance are detected. Any now known or later developed detection may be used. The detected data may be scan converted, remain formatted in the scan format (e.g., polar coordinate), be interpolated to a three-dimensional grid, combinations thereof, or be converted to another format. The detection and/or format conversion are done by separate devices, but may be implemented by the processor 20.

The processor 20 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for determining a path for high intensity focused ultrasound. The processor 20 is a single device or multiple devices operating in serial, parallel, or separately. The processor 20 may be a main processor of a computer, such as a laptop or desktop computer, or may be a processor for handling some tasks in a larger system, such as in an imaging system.

The processor 20 is operable to determine a bend of the blanket between the arrays as a function of output of the fiber optic sensor 15. The fiber optic sensor 15 outputs measurement values, position indications, or other information. The processor 20 receives the output and determines the locations of the arrays. The output may indicate the locations, so the determination is performed by receipt. In other embodiments, the processor 20 receives measurements for the position and/or orientation of the fiber optic sensor 15. Given the known locations of the arrays along the fiber optic sensor 15, the positions of the arrays are determined.

In another embodiment, the processor 20 determines the positions of the arrays using data correlation. The position determined from the fiber optic sensor 15 indicates the relative positions of the arrays at a given accuracy. A finer positioning is determined by correlation. The search region and/or step size for correlating different data sets representing overlapping regions are set based on or around the fiber optic sensor-based position. For example, the data is shifted from an initial position determined by the fiber optic sensor 15. Any number of translations and/or rotations may be used. Any tolerance, such as twice the spatial resolution of the fiber optic sensor-based position, may be used. For each shifted position, a level of similarity is determined, such as by cross-correlation or minimum sum of absolute differences. The shift associated with the highest similarity indicates the aligned position.

The processor 20 may perform other acts. In the example embodiment shown in FIG. 2, the processor 20 determines one or more paths to be used for high intensity focused ultrasound. For example, scan lines appropriate or more desired for the therapy transmissions are determined. The origin of the therapy beam of the high intensity focused ultrasound is identified. The origin and the treatment region define a scan line for transmitting the beam to the treatment region. By moving the origin, different scan lines or paths are identified. The origin may be for different transducers and/or different locations on a same transducer.

The origin and path are determined as a function of any desired factor. In one embodiment, patient characteristics between origin options and the treatment region are used to select the desired origin or origins. The characteristics of the patent along the possible paths are determined from data received with the imaging transducer 14. The imaging data indicates tissue characteristics. The processor 20 uses image processing to determine the tissue characteristics. The data along the paths may be analyzed for variation (e.g., high intensity followed by very low intensity indicating bone or metal with acoustic shadow), lack of variation (e.g., no tissue boundary), threshold intensity (e.g., bone or metal), Doppler response (e.g., fluid region), or other information. Two or three-dimensional processes, such as filtering and classification, may be used to identify tissue regions, tissue type, or other tissue characteristic.

Any patient characteristic, such as tissue attenuation, tissue type or identity, bone structure, metal fragments (e.g., shrapnel, bullet, or medical equipment), fluid region, or tissue boundaries, may be used. For example, bone or metal are identified as the patient characteristic along or adjacent to one or more possible paths. Instead of attempting to transmit high intensity focused ultrasound through or by acoustically opaque or scattering objects, other paths without such obstructions are selected. For example, therapy transducers or origins on a same therapy transducer without an obstruction along the path are selected. The selected origin or origins avoid transmitting the high intensity focused ultrasound through the bone or metal. The selected origin is from one of a plurality of therapy transducers or origins on a same transducer and not from another of the plurality of therapy transducers or origins on the same transducer. Paths with more fluid may be selected, since fluid may be better able to disperse any heat generated by even the distributed or out of focus high intensity ultrasound. A path through or by heat sensitive tissue may not be selected. Paths associated with less attenuation due to distance and/or tissue type may be selected. Paths with less scattering, such as with fewer tissue boundaries, may be selected.

The processor 20 may determine a power, frequency, or other characteristic of the transmitted high intensity focused ultrasound. The patient characteristic between the origin and the treatment region is used to set the power. Greater attenuation due to distance or tissue type may be accounted for by increasing the power. Greater scattering may be accounted for by increasing the power. The frequency may adapt depending on the type of transducer, depth, attenuation along the path, or other characteristic.

The memory 22 stores the ultrasound data for image processing. Alternatively or additionally, the memory 22 stores instructions for programming the processor 20 for position determination. The instructions for implementing the processes, methods and/or techniques discussed above are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

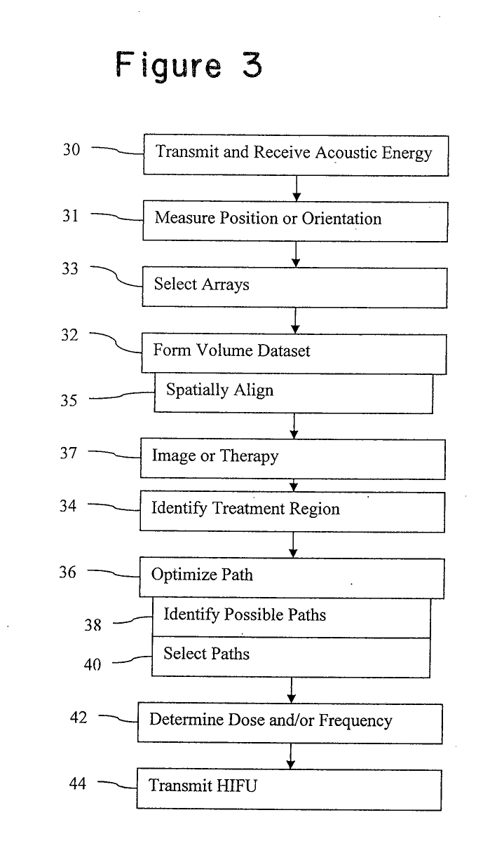

FIG. 3 shows a method for determining relative position of ultrasound scans. The method uses the system 10 of FIG. 1, the blanket 24 of FIG. 2, the blanket 24 of FIG. 7, different transducers, different conformal arrays and/or different systems. The acts are performed in the order shown or a different order. Additional, different, or fewer acts may be used. For example, the method is performed without acts 33-44.

In act 30, different regions are scanned. For example, the blanket ultrasound device or a conformal array is placed on the patient. The arrays conform to the contour of the patient. The arrays face in a same or different direction. For example, the blanket 24 flexes or expands between the arrays. The arrays of the ultrasound device or conformal array scan different, but overlapping regions of the patient. For example, different overlapping volumes are scanned with two-dimensional arrays. A sector or other format scan may provide more overlap than a linear scan.

To scan, acoustic energy is transmitted along a plurality of scan lines, and echoes are received in response to the transmission. The received echoes are converted into received electrical signals. The transmission and reception are performed for imaging, therapy, and/or testing possible paths.

The scan lines are formatted for scanning a plane or volume regardless of the possible paths. In one embodiment, a dataset representing a three-dimensional volume is formed by transmitting and receiving. The dataset is formed by scanning an entire volume. Alternatively, different scans of overlapping volumes are performed, and the overlapping volumes are combined. Different transducers scan different, but overlapping volumes. The acquisition is triggered and the multiple volumes are streamed to an external or post-processing processor.

Alternatively, the scan lines correspond to possible treatment paths. For example, the transmit and receive beams are formed along scan lines intersecting the region to be coagulated and from available sources of the high intensity focused ultrasound. One or multiple arrays may be used to form the beams along the desired scan lines. Previous imaging or other sensing may be used to determine the location of the region to be treated relative to the transducer or transducers.

In act 31, the positions of the scans are measured. A relative position of one transducer to another transducer is measured as a function of light intensity. The position of the transducer for each scan is sensed for aligning. For example, a flexible fiber optic sensor (e.g., flexible localization strip) determines relative position and orientation along the flexible sensor. The location is along a line, in a plane, or in three dimensions. The fiber optic sensor has electronic output to determine the spatial location of each ultrasound image voxel to be determined relative to each other. Absolute position sensing may be used, such as positioning one end or portion of the fiber optic sensor at a fixed, common, or known location. Other portions of the fiber optic sensor connect to the transducers. Magnetic, optic, gravity sensor, accelerometer, gyroscope, optical pattern recognition, infrared, radio frequency, or other position sensors may additionally indicate relative or absolute position.

The ultrasound imaging system determines the spatial relationship of the voxels or data samples to the transducer, and the transducer position sensing provides the relative or absolute position of the transducer or transducers. This allows every voxel of each sonographic acquisition to be assigned a spatial position. During acquisition, the positions and/or orientations of the transducers are sensed. The transducer position at each acoustic window is determined for spatially aligning the resulting acquisition data.

The position information is a position, orientation, or position and orientation of the transducer. The rotation between the first and second transducers is measured with the fiber optic sensor. Rotation and twist, or just twist, may be measured in other embodiments. The position information corresponds to one or more degrees of freedom or sensing, such as sensing 6 degrees of freedom (three translational axes and three rotational axes). The position information is sensed at the transducer or based on signals sent from the transducer.

In act 33, different arrays are selected for imaging or therapy. Some of the arrays may not be adjacent to the patient for scanning. For example, FIG. 7 shows three array panels 25 being folded over and not part of the cuff around the patient. These panels 25 are not positioned adjacent to the patient for scanning. The array panels 25 to be used for scanning are selected by identifying bending or rotation associated with non-use. For example, the light intensity shows a more drastic bend or extension away from the other panels 25, indicating the panels 25 to be used and not used for scanning. The array panels 25 not needed are identified to avoid scanning. All of the arrays may be used without selection in other embodiments.

In act 32, a volume dataset is formed. The volume dataset may be formed by scanning a volume with an array, or by combining datasets representing different volumes. For example, different arrays of a conformal array scan different, but overlapping volumes. The data from the different scans are combined together to form an extended volume and/or data representing an overlapping region. Alternatively, a planar dataset is formed of data representing one or more planes.

To form the dataset, the data from different scans is spatially aligned in act 35. In one embodiment, a stitching or “mosaicking” operation combines different volumetric datasets. For example, a first volume is expanded or added to with each new volumetric acquisition, while assuring insertion of the new information at the correct spatial position. In one embodiment, an ultrasound blanket device performs an initial acquisition, taken as reference. Then, additional volumes are acquired for combination.

The overlapping volumes are aligned. Position sensors, data correlation, or combinations thereof are used to determine the relative spatial position of the overlapping volumes. For correlation, speckle or other features may be used. In one embodiment, power Doppler information is segmented to identify one or more surfaces in each data set. The surfaces are then correlated by searching different rotations and/or translations. The relative position with the highest or sufficient correlation indicates the proper alignment. Cross-correlation, minimum sum of absolute differences, or other correlation may be used.

In other embodiments, B-mode data is used for alignment. In another embodiment, the power Doppler-based alignment is refined by further B-mode alignment. The power Doppler provides a lower resolution alignment, and the B-mode provides a higher resolution alignment. Features, speckle, segmentation, or other processes are used for B-mode alignment. For example, B-mode data with or without spatial filtering is correlated without specific feature extraction. Any search technique, such as set searching, numerical optimization, or coarse-fine, may be used for correlation-based spatial alignment.

In yet another embodiment, position sensor information or known spatial limitations of the relative position of the transducers (e.g., semi-rigid connection between transducers) is used to limit the search space for correlation. For example, data from scans of different regions are spatially aligned based on the relative position of the scans as measured by a fiber optic sensor. The fiber optic sensor indicates the difference in space of the transducer for each scan. The difference indicates a relative alignment. The alignment is further refined or a higher resolution alignment is determined by correlation. The fiber optic sensor provides an initial alignment to minimize the amount of searching needed with data correlation. By searching around at the initial alignment position, the number of translations and/or rotations to identify a better match is reduced.

In other embodiments, the alignment information from the fiber optic sensor alone is used for spatial alignment. Using the bending information and the known location of the imaging tiles/transducers, the datasets may be aligned for 3D stitching.

In other embodiments, different ones or combinations of data correlation and position sensors are used for translation and orientation. Without any bending estimation, the spatial alignment by data correlation iterates through a larger series of angles in order to match two partial volumes. The difficulty is amplified where the distance between two arrays can vary because of the elastic material linking the acoustic arrays. The spatial alignment depends on relative position (x, y, z) between two tiles, ultrasound beam angle, % of overlap between volumes, and angle between arrays. The fiber optic position sensor provides the angle between arrays. The remaining parameters are determined by scan format and data correlation. The fiber optic sensor providing the angle may reduce the time spent by the registration algorithm. Correlation may or may not be used for further enhancement of the angle position.

In act 32, the data of the aligned volumes is combined. The information is merged with the previous scan, based on the known locations of the transducers or volumes. Any combination may be used, such as selecting a datum for each spatial location from available datasets, averaging, weighted averaging to avoid combination artifacts, or interpolation. The aligned and combined volumes provide a larger three-dimensional volume.

In act 37, the compounded data set is used for imaging, therapy, or both. The volume dataset may be used for three-dimensional position determination, to generate cut planes, such as multiplanar reconstruction imaging, volume rendering, surface rendering, or other imaging. For example, a cut plane, which intersects and is co-axial with a vessel, is formed for identifying a region to be treated. The physician may perform effective diagnosis using images based on a single volume. Non-experts may be provided with an image of a large or extended region without requiring expert acquisition of data.

Acts 34, 36, 38, 40, 42, and 44 provide one example of using the compounded dataset for application of therapy. In act 34, the region to be coagulated or treated is identified. Manual, automatic, or semi-automatic identification is used. For example, the user selects a point in different views as indicating the location of a bleeding vessel. The geometric relationship of the different views may provide an indication of a location in a volume for treatment. As another example, a processor identifies the region for vascular closure of an internal hemorrhage. An image process is performed to identify the leakage of a vessel. The volume dataset or other data representing the possible location of internal bleeding is processed. Any type of data may be processed, such as ultrasound, CT, X-ray, or MRI.

In one embodiment, ultrasound data representing the volume, such as acquired with a blanket ultrasound device, is used to localize the bleeder with a processor. For example, Doppler information shows a flow pattern associated with bleeding. As another example, B-mode data shows a tear or hole in a vessel wall using boundary detection and high pass filtering of the boundary. In another example, power Doppler data is segmented to identify the locations of flow within a volume. Using skeletonization, the centerlines of any flow and bifurcations result. The bifurcation represents either a vessel branch or bleeding. Spectral Doppler gates are positioned in the vessel before a bifurcation and at each branch of the bifurcation. Alternatively, a spectral Doppler gate is positioned at only one branch. The systole and diastole spectral response patterns for vessel flow and bleeding are different. Bleeding has less heart cycle variation. By examining a pattern or by comparing patterns, the processor may determine whether a bifurcation is a hemorrhage. In yet another example, acoustic force radiation is used to vibrate a vessel wall. Differences in vibration results may indicate a location of bleeding.

In act 36, a path to the region to be coagulated is optimized for high intensity focused ultrasound. The path is optimized by selecting a better path than others.

A plurality of possible scan lines is determined in act 38. For example, scan line origins based on the available transducers for HIFU and/or based on the available or sampled locations on the face of one or more transducers for HIFU are included in the set of possible paths. For example, two or more scan lines are identified as originating from a respective two or more separate therapy transducers. Other limitations or inclusions may be used to determine the set of possible paths. Each path is a straight line from the origins to the region to be treated within the patient, so corresponds to a scan line or beam volume for the transmission of an ultrasound treatment beam.

The optimization provides for one or more paths. For example, multiple paths may be used to distribute a heat load on skin or tissue not to be treated. A single path may be used.

The spatial relationship of the HIFU transducers to the location to be treated is known or measured. For example, each HIFU transducer is rigidly mounted to an imaging transducer. The alignment of data from the different imaging transducers and the use of imaging data to identify the treatment region provide the spatial relationship of the HIFU transducer to the treatment region. As another example, the relative position of the HIFU transducer to the imaging transducer is measurable, such as with a strain gauge or other sensor. In another example, acoustic reflections from the HIFU transducer indicate the spatial relationship of the HIFU transducer to an imaging transducer. Combinations of these techniques or other techniques may be used.

In act 40, one or more of the possible paths are selected. All or a subset of one or more of the possible paths are selected. The optimization is a function of the received signals. Signals received from scanning the treatment region and around the treatment region indicate the path or paths to be used.

In one embodiment, the possible paths are tested by transmitting along each possible path. Acoustic energy, such as for imaging, is transmitted along the scan lines of each possible path. The signals representing the returning echoes along the scan lines are examined to identify the optimum path or paths.

In another embodiment, the possible paths are identified through a volume where the data representing the volume is acquired without regard to the possible paths or aligned with the possible paths. The scan lines for acquiring the volume dataset may or may not correspond to the possible paths. Since data is acquired for the volume, at least a portion of each possible path has data representing the path. Rays corresponding to the possible paths are cast through or positioned within the volume. The rays are from the available sources of the HIFU through the volume to the region to be treated. The data along the rays may be examined for optimization.

Once the overlapping volumes are stitched together, it is possible to retrieve any voxel (data from the dataset). For a given voxel along the ray, the imaging transducers which contributed data for the combination (e.g., for selection or averaging) are identified. A list of ultrasound imaging transducers involved for any particular point in the medical volume is retrieved. The list of imaging transducer may be used to rule out HIFU transducers associated with imaging transducers that did not contribute data to each voxel along a possible path. For example, an obstruction may result in an imaging transducer not providing data for a location. Alternatively, the data without consideration to source is examined for selection.

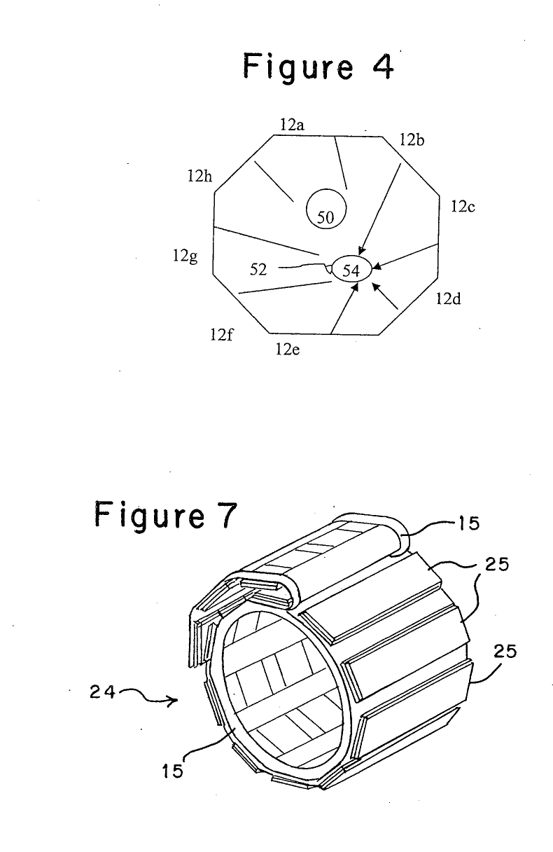

The paths are selected to avoid an acoustic obstruction, a heat sensitive region, a high attenuation region, scatterers, or combinations thereof. The characteristics for the selection are provided by the data along the paths. FIG. 4 shows HIFU transducers 12a-h surrounding a treatment region 54. Adjacent the treatment region is a bone 50 and a piece of metal 52, such as associated with hemorrhaging due to shrapnel in a leg. Possible paths are represented by lines from each HIFU transducer 12a-h towards the treatment region 54. For HIFU transducers 12a, and 12f-h, the lines intersect or are close to the metal 52 or bone 50. To provide the desired power for coagulation, the HIFU should not be transmitted into an obstruction. To prevent heating material that may cause further damage (e.g., the metal 52), paths intersecting or close to the material are not selected. The paths free of obstruction are selected, such as from HIFU transducers 12b-e.

Other or different criteria may be used. For example, tissue along a path is heat sensitive, so the path is not selected. As another example, a path passes through more fluid and/or tissues with less attenuation, so is selected. In another example, paths with shorter distances are selected to minimize attenuation, allowing transmission of less power to provide the same power absorption at the treatment region.

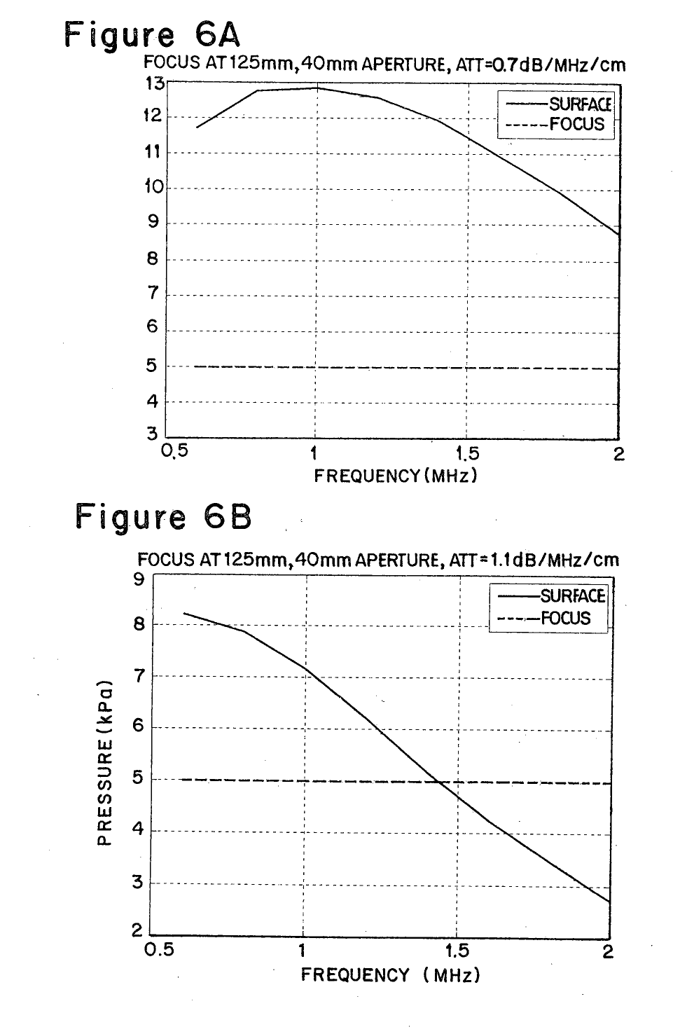

The tissue characteristics (e.g., obstructions) may be detected from the data. Image processing may identify a type of tissue, providing indication of attenuation coefficient. Intensity of reflection, change in intensity as a function of depth or other data analysis indicates obstructions. For example, ray casting in a volume identifies imaging transducers contributing to a voxel at the treatment region. If an imaging transducer did not contribute to the voxels at the treatment region, an obstruction may be indicated. As another example, FIG. 5 shows two rays through a volume or along a plane. The intensities of the voxels along the two rays are shown by voxel and as an analog wave. The intensity variation, peaks, minimum, or other characteristics may indicate the path as desirable or not.

In act 42, the characteristics of the HIFU transmit beam or beams are determined by a processor, by a user, or combinations thereof. The characteristics include power, frequency, combinations thereof, and/or other characteristics (e.g., duration, sequence, or pulse repetition interval). The determination may be a function of the selected paths. For example, higher power pulses may be transmitted for a fewer number of paths. The determination is a function of the desired therapy or amount of power to be delivered in a specific period to cause coagulation or provide treatment. Any now known or later developed dosage considerations may be used for the HIFU beam or beams.

In one embodiment, the power and frequency of the high intensity focused ultrasound is determined, at least in part, as a function of a characteristic of the path. For example, the frequency of the high intensity ultrasound adapts as a function of depth from the HIFU transducer to the treatment region, attenuation characteristic along the path, or combinations thereof. The optimum HIFU frequency depends on the target depth, attenuation constant, the transmit transfer function of the transducer, and any limiting factor. Limiting factors may include, for example, maximizing the power absorption at the target depth or minimizing the power absorption at the skin. The frequency at which the acoustic intensity is highest may not be the optimum HIFU frequency because of the frequency dependence of the acoustic absorption. A desired or optimum HIFU frequency may be calculated given the target depth, and the tissue type between the target and the transducer. Image processing, thresholding, or other technique may be used to distinguish tissue type. For example, fluid, soft tissue and bone tissue types or structures may be distinguished. More subtle distinctions between types of soft tissue may be made. The different types are associated with different acoustic attenuation.

Tissue heating is achieved by absorption of acoustic power. Acoustic absorption is proportional to an attenuation coefficient. Higher attenuation provides higher acoustic power absorption and heat generation. Attenuation and absorption increase with frequency, so it is desirable to use higher frequencies for heating. However, higher propagation attenuation at higher frequencies means shallower penetration depth. There is a trade-off between penetration depth and frequency, and heat. For a given depth of the treatment region, there may be a better frequency at which maximum power deposition (so ΔT) is achieved.

For a plane wave, the pressure at a depth z is related to the pressure at the surface of the transducer with the following equation:

P(z)=P₀·e^{-α·f}^{^{k}}^{·z},

where P (z) is the pressure amplitude as a function of depth (z), P₀ is the pressure at z=0, and α·f^{k} is the frequency dependent tissue attenuation constant (k usually takes a value between 1 and 2 depending on the tissue and α is an attenuation coefficient). The acoustic power absorbed by the tissue, L (z), is then calculated as:

$$L ( z ) = \frac{α \cdot {f}^{k}}{{Z}_{0}} {P}^{2} ( z ) .$$

Absorbed power is proportional to the frequency dependent attenuation constant. The frequency where maximum acoustic power absorption is shown as:

$${f}_{max} = {( \frac{1}{2 \cdot α \cdot z} )}^{\frac{1}{k}} .$$

The optimum frequency depends on the depth and attenuation constant. Note that, this calculation is for simple plane waves and is intended to show the dependence of the optimum frequency on the depth and attenuation constant. HIFU beams may be transmitted as a plane wave or with a greater focus. For a transducer with transmit beamforming and a non-uniform tissue type between the transducer and the target (e.g., non-uniform attenuation constant), the optimum frequency may be calculated numerically.

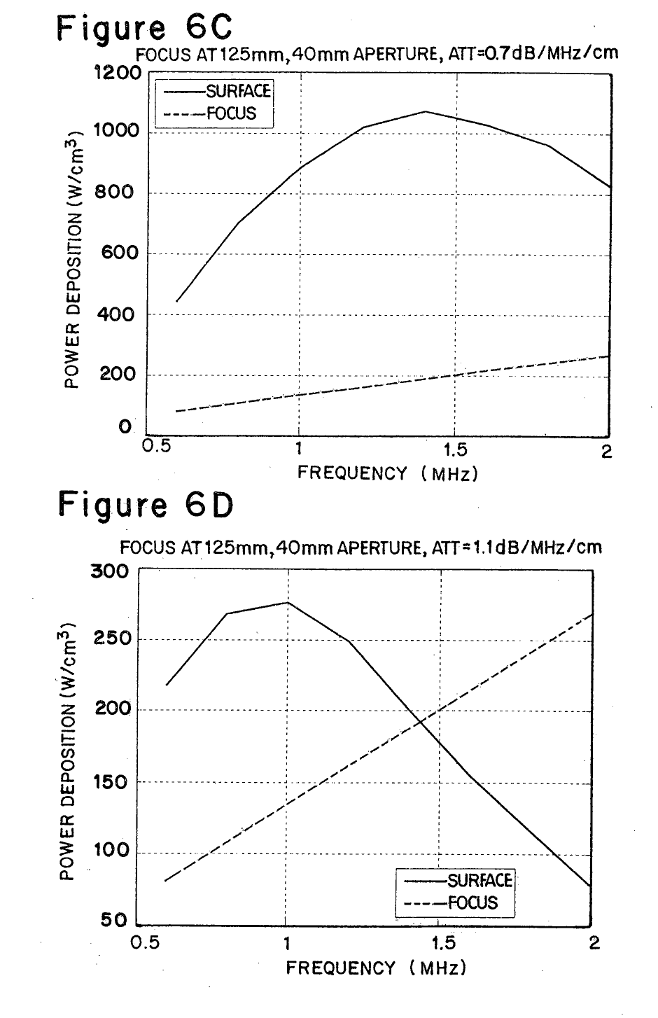

For example, a hypothetical 2D transducer array can generate 5 kPa at its surface independent of the frequency. The transducer has an aperture of 40 mm by 40 mm. FIGS. 6A and 6B show the pressure at the target depth of 125 mm together with the pressure at the surface for 0.7 dB/MHz/cm attenuation (k=1) and for 1.1 dB/MHz/cm attenuation (k=1), respectively. The skin surface is represented by a straight line and the focus is represented by the curve in the Figures of FIG. 6. FIGS. 6C and 6D show the power absorbed by the tissue at the target depth of 125 mm and at the surface for 0.7 dB/MHz/cm attenuation (k=1) and for 1.1 dB/MHz/cm attenuation (k=1), respectively. Comparing FIGS. 6A and 6B with 6C and 6D shows that the frequency of maximum power absorption is not the same as the frequency of maximum acoustic intensity (square of acoustic pressure).

The absorption depends on the attenuation constant. Knowing an average tissue attenuation or the tissue attenuation profile between the target and the transducer may increase the accuracy of optimum frequency calculation. The attenuation constant of different detectable tissue types may be determined and incorporated into the algorithm.

FIGS. 6E and 6F reveal that the operating frequency should be chosen to avoid heating the skin more than the target tissue. Depending on the limiting factor (power absorption at the target depth or power absorption at the skin), the optimum HIFU frequency may be different.

This example shows the case for a transducer with a flat spectral bandwidth. The transducers may have a transfer function affecting the optimum frequency. For a given transducer whose transmit transfer function is known and a given target depth from the transducer, if the attenuation constant is known in the tissue between the transducer and the target, then the optimum HIFU frequency can be calculated numerically. The HIFU optimum frequency can be calculated automatically. The frequency may depend on fewer or additional factors, such as just on the attenuation.

In addition or as an alternative, the power dose of the high intensity ultrasound along each of the selected paths is determined. The power dose may be determined a function of tissues along the path, distance from the transducer to the treatment region along the path, number of paths in the subset, frequency of the transmission, combinations thereof, or other factors. For example, different tissue types provide different attenuation. The different attenuation of the treatment region and the regions between the treatment region and the transducer may alter the power delivered for treatment. Greater attenuation along the path may result in a higher power dose transmitted from the transducer. Greater absorption at the treatment region may result in a less power dose transmitted from the transducer. The power dose is altered by changing frequency, amplitude, or number of cycles of the transmitted waveforms.

The specific tissue types may be identified. Alternatively, the intensity of the echoes or data along the path may indicate tissue characteristics. For example, FIG. 5 shows the intensities along two paths. By collecting the intensities along the paths, the amount of power to reach that particular anatomical point with a desired power level is calculated. The average intensity, sum of intensities, or intensity profile may correlate with attenuation. Other functions may be used to determine power dose.

In act 44 of FIG. 3, the high intensity ultrasound is transmitted along one or more of the paths of the selected subset of possible paths. The high intensity focused ultrasound is transmitted along the selected rays or scan lines. The HIFU transmit beam has a greater cumulative power than the imaging acoustic energy. For a given beam, the power of the HIFU may be greater than used for the imaging beams. If sufficient paths are provided, the HIFU power for a given beam may be less due to distribution of the transmitted power. Since the HIFU beams have the same or adjacent focus, the delivered power at the treatment region is greater than from an imaging scan.

The HIFU beams are transmitted along each path at a same or substantially same time so that the power delivered at a given time at the treatment region is sufficient. Sequential transmission along different or the same paths or combinations of sequential and simultaneous may be used to provide the desired total power, temporally distributed power, and/or spatially distributed power.

The ultrasound energy is focused at the treatment region. If sufficient energy is radiated to the treatment region, cells located in the focal volume may be rapidly heated while intervening and surrounding tissues outside the focus are spared the same level of heating. Surrounding tissues are unaffected or affected less in the unfocused portion of the ultrasound beam because the energy is spread over a correspondingly larger area. The transmitted HIFU pulses have the determined frequency, power dose, or other characteristic.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for determining relative position of ultrasound scans, the method comprising:
scanning a first region with a first ultrasound transducer;
scanning a second region with a second ultrasound transducer;
measuring a relative position of the first ultrasound transducer with the second ultrasound transducer as a function of light intensity; and
spatially aligning data from the scanning of the first region with data from the scanning of the second region, the spatially aligning being a function of the relative position.

2. The method of claim 1 wherein scanning comprises scanning the first and second regions as first and second volumes, respectively.

3. The method of claim 1 wherein scanning comprises scanning with a blanket transducer including the first and second ultrasound transducers flexibly connected together.

4. The method of claim 1 wherein measuring the relative position comprises measuring rotation between the first and second transducers with a fiber optic sensor.

5. The method of claim 1 wherein measuring the relative position comprises measuring relative rotation and twist between the first and second transducers.

6. The method of claim 1 wherein spatially aligning comprises spatially aligning as a function of the relative position and correlation of the data from the scanning of the first and second regions, the first and second regions overlapping.

7. The method of claim 6 wherein spatially aligning as a function of the relative position and the correlation comprises aligning based on the correlation with searching in the correlation being limited by the relative position.

8. The method of claim 1 wherein the first and second transducers are part of a conformal array of a plurality of transducers;
further comprising:
selecting at least one of the plurality of transducers for imaging or therapy as a function of the light intensity.

9. The method of claim 1 further comprising:
ultrasound imaging, applying ultrasound therapy, or both as a function of the spatially aligned data.

10. The method of claim 1 further comprising:
identifying a plurality of possible paths for high intensity ultrasound from one or more of the first and second transducers to a treatment region within a patient;
selecting a subset of the plurality of possible paths as a function of ultrasound response; and
transmitting the high intensity ultrasound along the paths of the selected subset.

11. A system for determining relative position of ultrasound scans, the system comprising:
a plurality of arrays;
a blanket flexibly connecting the plurality of arrays;
a fiber optic sensor connected with or adjacent each of the arrays of the plurality; and
a processor operable to determine a bend of the blanket between the arrays as a function of output of the fiber optic sensor.

12. The system of claim 11 wherein the plurality of arrays comprises at least one therapy transducer and at least one imaging transducer.

13. The system of claim 12 wherein the at least one therapy transducer is operable to transmit high intensity focused ultrasound;
wherein the at least one imaging transducer is operable to transmit acoustic energy for imaging; and
wherein the processor is operable to determine an origin of a beam of the high intensity focused ultrasound relative to a treatment region, the origin determined as a function of patient characteristics between origin options and the treatment region, and the processor being operable to determine the patient characteristics and origin as a function of data received with the imaging transducers.

14. The system of claim 11 wherein the fiber optic sensor is operable to detect bend and twist along a length of the fiber optic sensor.

15. The system of claim 11 wherein the fiber optic sensor is within the blanket.

16. The system of claim 11 wherein the blanket and the plurality of arrays comprise a conformal array of ultrasound transducer elements.

17. A medical ultrasound transducer comprising:
a conformal array of transducer elements;
fiber optic strands connected with the conformal array; and
a sensor operable to determine bend of the conformal array as a function of light transmitted in the fiber optic strands.

18. The transducer of claim 17 wherein the conformal array of transducer elements comprises a plurality of multidimensional arrays flexibly connected together.

19. The transducer of claim 18 wherein the plurality of multidimensional arrays comprise at least one imaging array and at least on therapy array.

20. The transducer of claim 18 wherein the conformal array comprises a cuff.

21. A system for determining relative position of ultrasound scans, the system comprising:
an ultrasound array of elements;
a fiber optic sensor connected with or adjacent to the ultrasound array and connectable at another location; and
a processor operable to determine a position of the ultrasound array as a function of output of the fiber optic sensor.
